(19) United States
(12) Patent Application Publication
(10) Pub. No.: US 2010/0069479 A1
(43) Pub. Date: Mar. 18, 2010

(54) NEURODEGENERATIVE DISEASE TREATMENT USING JAK/STAT INHIBITION

(75) Inventors: Jun Tan, Tampa, FL (US); Frank Fernandez, Tampa, FL (US); Nan Sun, Tampa, FL (US); Roland D. Shytle, Largo, FL (US); Jared Ehrhart, Tampa, FL (US)

Correspondence Address: SMITH HOPEN, PA, 180 PINE AVENUE NORTH, OLDSMAR, FL, 34677, US

(73) Assignee: UNIVERSITY OF SOUTH FLORIDA, Tampa, FL (US)

(21) Appl. No.: 12/552,715

(22) Filed: Sep. 2, 2009

Related U.S. Application Data
(63) Continuation of application No. PCT/US2008/055646, filed on Mar. 3, 2008.
(60) Provisional application No. 60/892,619, filed on Mar. 2, 2007.

(51) Int. Cl. A61K 31/352; C12N 5/00; A61P 25/00

(52) U.S. Cl. 514/456; 435/375

(57) ABSTRACT

The invention relates to treatment of neurodegenerative diseases with JAK/STAT pathway inhibitors to eliminate extracellular cell signaling events leading to cell cycle abrogation and/or apoptosis. Primary neurons were administered neurotoxic proteins, such as gp120, Tat, or gp120 and Tat, with or without IFN-γ added, resulting in neuronal death, and simulated neurodegenerative diseases. The neurodegenerative disease is treated using a JAK/STAT pathway inhibitor, including (—)-epigallocatechin-3-gallate (EGCG), to modulate JAK1 or STAT1 phosphorylation, resulting in resistance to gp120 or Tat neurotoxicity. The invention may be used to treat neurons afflicted with HIV-associated Dementia, multiple sclerosis, Alzheimer's Disease, Parkinson's Disease, amyotrophic lateral sclerosis, or Pick's Disease, and may act in conjunction with antiviral treatment, like HAART.

### CROSS REFERENCE TO RELATED APPLICATIONS

This application is a continuation of prior filed International Application Ser. Number PCT/2008/055646 filed Mar. 3, 2008, which claims priority to U.S. Provisional Patent Application Number 60/892,619, entitled, “Treatment for HIV Dementia”, filed Mar. 2, 2007, the contents of which are herein incorporated by reference

### FIELD OF INVENTION

This invention relates to human immunodeficiency virus treatment. Specifically, the invention involves mitigating the neurotoxic effects of HIV-1 proteins.

### BACKGROUND OF THE INVENTION

Epidemiologic studies indicate that approximately 60% of human immunodeficiency virus (HIV)-1-infected patients suffer some form of related neuropsychiatric impairment (H. Ozdener, Molecular Mechanisms of HIV-1 Associated Neurodegeneration, J. Biosci., 30, 391-405, 2005; A. Stephanou, Role of STAT-1 and STAT-3 in Ischemia/Reperfusion Injury, J. Cell Mol. Med., 8, 519-525, 2004), characterized by cognitive, motor, and/or behavioral symptoms. HIV-associated dementia (HAD) is a metabolic encephalopathy that represents the most severe form of HIV-related neuropsychiatric impairment (P. Shapshak, et al., Elevated expression of IFN-gamma in the HIV-1 Infected Brain, Front. Biosci., 9, 1073-1081, 2004), with an average survival after diagnosis of 6 months (A. Nath, et al., Transient Exposure to HIV-1 Tat Protein Results in Cytokine Production in Macrophages and Astrocytes, J. Biol. Chem. 17098-17102, 1999). During early stages of HIV infection, virus invades the central nervous system (CNS) tissue from peripheral cell populations, including infected monocytes and macrophages and T-cells. Through this process, HIV establishes a viral reservoir in the CNS early after primary infection which is resistant to highly activated antiretroviral therapy (HAART; S. T. Melton, et al., Pharmacotherapy of HIV Dementia, Ann. Pharmacother., 31, 457-473, 1997). Later in the symptomatic phase of HAD, commonly coinciding with CD4+T-cell depletion below 200 cells/mm³, the virus is sustained in the CNS primarily by resident microglia and macrophages that invaded from peripheral tissues. These cells serve as both viral factories and mediators of inflammatory events, resulting in neuropathology and related neuropsyciatric impairment (S. Aquaro, et al., Human Immunodeficiency Virus Infection and Acquired Immunodeficience Syndrome Dementia Complex: Role of Cells of Monocyte-Macrophage Lineage, J. Neurovirol., 11, 58-66, 2005; M. Kumar, et al., HIV-1 Infection and its Impact on the HPA Axis, Cytokines, and Cognition, Stress, 6, 167-172, 2003, Shapshak, et al., 2004; H. Xiong, et al., HIV-1 Infected Mononuclear Phagocyte Secretory Products Affect Neuronal Physiology Leading to Cellular Demise: Relevance for HIV-1-Asscoiated Dementia, J. Neurovirol., 6, S14-S23, 2000). Pathologic CNS immune dysfunction has been widely explored in many past studies of microglia, the primary host cells for HIV-1 in the CNS (G. Garden, et al., Caspase Cascades in Human Immunodeficiency Virus-Associated Neurodegeneration, J. Neurosci., 22, 4015-4024, 2002; S. Koenig, et al., Detection of AIDS Virus in Macrophages in Brain Tissue from AIDS Patients with Encephalopathy, Science, 233, 1089-1093, 1986).

In HIV infection, a CNS viral reservoir is initiated early after infection within the central nervous system. In later stages of HIV, the neuronal damage and cognitive impairment found in HAD surface. The specific components leading to neurological dysfunction in HAD remain unclear, with current studies aimed at differentiating and characterizing individual disease mechanisms involved in chronic inflammatory activation of immune effector cells and HIV protein-induced dysfunction of neurons, ultimately resulting in neuronal cell death.

In HAD, neurons are not killed by direct viral infection but rather viral proteins released from infected CNS mononuclear cells may directly kill neurons or render them susceptible to death signaling. Clearly viral proteins can bind to cell surface receptors such as CXCR4 and N-methyl-o-aspartate receptors. Thus HIV-1 proteins gp120 and Tat may trigger neuronal apoptosis and excitotoxicity resulting from altered cellular intracellular calcium concentrations and mitochondrial dysfunction (M. P. Mattson et al., Cell Death in HIV Dementia, Cell Death Differ. Suppl., 1, 893-904, 2005). Inflammation and proinflammatory soluble factors also play important roles in the pathogenesis of HAD. Increasingly, studies point to the central roles played by reactive immune cells including macrophages and microglia in the generation and progression of many disease mechanisms implicated in the pathology of HAD (Aquaro et al., 2005), as well as other neurodegenerative diseases.

HIV-1 rarely infects neurons (W. Li, et al., Molecular and Cellular Mechanisms of Neuronal Cell Death in HIV Dementia, Neurotox. Res., 8, 119-134, 2005), thus focusing investigations on the neurotoxic effects of excreted viral proteins, including HIV-1 gp120 and Tat. HIV-1 protein gp120 is a viral envelop protein that binds to CD4 receptors and assists in viral fusion to host cells, whereas Tat is a viral transcription regulator. Previous investigations have demonstrated cause and effect relationships between production of HIV-1 proteins gp120 and Tat, and neuronal damage (Li et al., 2005; Mattson et al., 2005; Nath et al., 1999). Capable of directly inducing neuron damage through apoptosis, gp120 and Tat may be enhanced by cytokine-mediated signaling. Cytokines IFN-γ, TNF-α, and IL-1β have been shown to augment the neurotoxicity of gp120 (F. Peruzzi, et al., Cross Talk Between Growth Factors and Viral and Cellular Factors Alters Neuronal Signaling Pathways: Implication for HIV-Associated Dementia, Brain Res. Rev., 50, 114-125, 2005). A similar mechanism has been suggested for Alzheimer's disease, where IFN-γ has been demonstrated to augment neuronal death in response to amyloid-beta (C. Bate, et al., Interferon-gamma Increases Neuronal Death in Response to Amyloid-betal-42, J. Neuroinflammation, 28, 1-7, 2006). Studies investigating the neurotoxic effects of IFN-γ implicated members of the JAK and STAT families (M. R. Heitmeier et al., Prolonged STAT1 Activation is Associated with Interferon-gamma Priming for Interleukin-1-Induced Inducible Nitric-Oxide Synthase Expression by Islets of Langerhans, J. Biol. Chem., 274, 29266-29273, 1999; K. Y. Lee et al., Loss of STAT1 Expression Confers Resistance to IFN-gamma-Induced Apoptosis in ME180 Cells, FEBS Lett., 459, 323-326, 1999), and have implicated this Th1 cytokine in the pathophysiology of HAD (Benveniste, et al., 1994). IFN-γ binding to its receptor activates Janus associated kinases (JAKs), which phosphorylate tyrosine residues on the intracytoplasmic side of the IFN-γ receptor leading to signal transducer and activator of transcription (STAT) proteins. Once activated, STAT dimerizes and migrates to the nucleus, thereby transcriptionally activating or repressing genes; a system known collectively as the JAK/STAT pathway (Heitmeier et al., 1999).

The JAK1/STAT1 interaction is extensively described in studies investigating apoptosis induced by ischemia/reperfusion in cardiovascular, CNS, and other tissues (Kumar et al., 1997; Stephanou, 2004). In neurons, STAT1 appears to be primed by ischemia/reperfusion and thus rendered more sensitive to IFN-γ receptor activation (Stephanou, 2004; Y. Takagi et al., STAT1 is Activated in Neurons After Ischemia and Contributes to Ischemic Brain Injury, J. Cereb. Blood Flow Metab., 22, 1311-1318, 2002). Occlusion of the middle cerebral artery resulted in rapid co-localization of STAT1 with TUNEL-positive neurons, thereby suggesting a role for STAT1 in cell apoptosis/death (Takagi et al., 2002). In normal cells, IFN-γ-mediated JAK/STAT1 activation is transient, lasting from several minutes to several hours.

One hypothesis to explain HAD suggests the JAK/STAT regulatory system of pro-inflammatory and apoptotic signaling is dysfunctional in HAD patients. The regulatory system is in a recurring state of inflammatory, cytokine-mediated apoptotic signaling, leading to widespread neuron damage (Lee, et al., 1999; Peruzzi, et al., 2005; Shapshak, et al., 2004). Previous studies support a role for JAK/STAT activation in the mediation of neuronal damage in HAD (E. N. Bovolenta, et al., Constitutive Activation of STATs Upon In Vitro Human Immunodeficiency Virus Infection, Blood, 94, 4202-4209, 1999) as well as stroke (Stephanou, et al., 2000). Further, HIV infection of the CNS induces marked increases in IFN-γ expression in CNS tissues (Shapshak et al., 2004).

Therefore what is needed is a treatment to comabt cell death caused by HIV viral attack on the brain and a treatment to limit or eliminate the neuortoxicity-enhancing effects of IFN-γ.

### SUMMARY OF INVENTION

The invention relates to treatment of neurodegenerative diseases with JAK/STAT pathway inhibitors to eliminate extracellular cell signaling events leading to cell cycle abrogation and/or apoptosis. The present invention may be used to treat neurons afflicted with HIV-associated Dementia, multiple sclerosis, Alzheimer's Disease, Parkinson's Disease, amyotrophic lateral sclerosis, or Pick's Disease. In one aspect of the invention, treatment alleviates HIV-associated dementia, and specifically dementia caused by HIV gp120 or Tat protein and is enhanced by IFN-γ. The ability of IFN-γ to enhance neuronal damage inflicted by HIV-1 proteins gp120 and Tat in mice is shown in vitro and in vivo; an effect associated with increased JAK/STAT1 signaling.

To study neurodegenerative disease, primary neurons were administered neurotoxic proteins, such as gp120, Tat, or gp120 and Tat. The administration of the proteins results in neuronal death, and simulates neurodegenerative diseases. IFN-γ was optionally added to the cells to further enhance the neuronal death, and also simulates some neurodegenerative diseases, like HIV-Associated Dementia.

The neurodegenerative disease is caused by cellular responses to activation of the JAK/STAT pathway, and in some embodiments the JAK1/STAT1 pathway. Thus, treatment of the disease entails using an effective amount of a JAK/STAT pathway inhibitor to modulate JAK1 or STAT1 phosphorylation, and preferably to modulate JAK1 phosphorylation. Cells were treated with JAK1 inhibitor or comprise STAT1-deficient neurons, resulting in resistance to gp120 or Tat neurotoxicity. The addition of IFN-γ, shown to enhance gp120 and Tat neurotoxicity, did not affect cells treated with JAK1 inhibitor or containing STAT1 deficiency.

In another aspect of the present invention, cells were treated with a polyphenol, preferably a catechin, and especially (—)-epigallocatechin-3-gallate (EGCG). EGCG. EGCG is a major constituent of green tea and EGCG modulates neuronal damage by inhibition of JAK/STAT1 activation. Importantly, EGCG treatment attenuated HAD-like neuronal injury mediated by HIV-1 proteins gp120 and Tat in the presence of IFN-γ in vitro and in vivo through JAK/STAT1 inhibition.

EGCG is preferably administered to a patient at a concentration between 5 μM and 40 μM, especially at a concentration of 20 μM. The treatment may acts as an adjuvant to an antiviral treatment, like HAART.

### BRIEF DESCRIPTION OF THE DRAWINGS

For a fuller understanding of the invention, reference should be made to the following detailed description, taken in connection with the accompanying drawings, in which:

FIG. 1 is a graph depicting IFN-γ enhancing neuronal injury induced by HIV-1 proteins gp120 or Tat, in vitro and in vivo. Primary cultured neuronal cells were treated with gp120 (250 ng/ml), Tat (250 ng/ml), IFN-7 alone or gp120 (250 ng/ml), Tat (250 ng/ml) in combination with IFN-γ (100 U/ml; IFN-γ/gp120/or IFN-γ/Tat) for 12 h. Cell cultured media were collected for LDH assay. Data are presented as the mean±SD of LDH. One-way ANOVA followed by post hoc comparison revealed significant differences between gp120 or Tat and HIV-1 gp120 or Tat plus IFN-γ (**p<0.001) for LDH release.

FIG. 2 is a blot depicting IFN-γ enhancing neuronal injury induced by HIV-1 proteins gp120 or Tat, in vitro and in vivo. Primary cultured neuronal cells were treated with gp120 (250 ng/ml), Tat (250 ng/ml), IFN-7 alone or gp120 (250 ng./ml), Tat (250 ng/ml) in combination with IFN-γ (100 U/ml; IFN-γ/gp120/or IFN-γ/Tat) for 12 h. Cell lysates were prepared for neuronal injury examination by Western blot analysis.

FIG. 3 is a graph depicting IFN-γ enhancing neuronal injury induced by HIV-1 proteins gp120 or Tat, in vitro and in vivo. Primary cultured neuronal cells were treated with gp120 (250 ng/ml), Tat (250 ng/ml), IFN-7 alone or gp120 (250 ng/ml), Tat (250 ng/ml) in combination with IFN-γ(100 U/ml; IFN-γ/gp120/or IFN-γ/Tat) for 12 h. Cell lysates were prepared for neuronal injury examination by Western blot analysis and results quantitated. Data are presented as Western blot band density ratios of Bc1-xL to Bax (n=3). One-way ANOVA followed by post hoc comparison revealed significant differences between gp120 or Tat and HIV-1 gp120 or Tat plus IFN-γ (**p<0.001) for the band density ratio of Bc1-xL to Bax.

FIG. 4 is a blot depicting IFN-γ enhancing neuronal injury induced by HIV-1 proteins gp120 or Tat, in vitro and in vivo. Primary cultured neuronal cells were treated with gp120 (250 ng/ml), Tat (250 ng/ml), IFN-7 alone or gp120 (250 ng/ml), Tat (250 ng/ml) in combination with IFN-γ (100 U/ml; IFN-γ/gp120/or IFN-γ/Tat) for 12 h. Bc1-xL and Bax protein levels in mouse brain homogenates were analyzed by Western blot.

FIG. 5 is a graph depicting IFN-γ enhancing neuronal injury induced by HIV-1 proteins gp120 or Tat, in vitro and in vivo. Primary cultured neuronal cells were treated with gp120 (250 ng/ml), Tat (250 ng/ml), IFN-7 alone or gp120 (250 ng/ml), Tat (250 ng/ml) in combination with IFN-γ (100 U/ml; IFN-γ/gp120/or IFN-γ/Tat) for 12 h. Western blot Data are presented as the mean±SD of Western blot band density ratios of Bc1-xL to Bax (n=8; 4 male/4 female). One-way ANOVA followed by post hoc comparison revealed significant differences between gp120 or Tat compared to gp120 or Tat plus IFN-'y for band density ratio of Bc1-xL to Bax (**P<0.001).

FIGS. 6(A) through 6(D) are microscopy images depicting IFN-γ enhancing neuronal injury induced by HIV-1 proteins gp120 or Tat, in vitro and in vivo. Primary cultured neuronal cells were treated with (A) PBS (10 l) (control), (B) gp120 (250 ng/ml), (C) IFN-7 or (D) gp120 in combination with IFN-γ (100 U/ml; IFN-γ/gp120) for 12 h. Mouse coronal, frozen brain sections were stained with NeuN. Marked neuronal damage was observed in the gp120 plus IFN-γ condition compared to controls. Similar results were also observed in the Tat plus IFN-γ condition (data not shown).

FIG. 7 is a graph depicting JAK/STAT1 signaling is critically involved in the IFN-γ mediated enhancement of HIV-1 gp120 and Tat-induced neuronal damage. Primary cultured neuronal cells were co-treated with IFN-γ (100 U/ml) and gp120 or Tat at 250 ng/ml in the presence of JAK inhibitor (50 nM) for 12 h. Cell cultured media was collected for LDH assay. Data are presented as mean±SD of LDH release (n=3). One-way ANOVA followed by post hoc comparison revealed significant differences between IFN-γ/gp120 or TFN-γ/Tat compared to JAK inhibitor/IFN-γ/gp120 or JAK inhibitor/IFN-γ/Tat (**P<0.001). Primary neuronal cells derived from STAT1-deficient mice were treated with gp120 or Tat at 250 ng/ml in the presence or absence of IFN-γ (100 U/ml) for 12 h. Cell cultured media and cell lysates from these cells were subjected to LDH assay.

FIG. 8 is a blot depicting JAK/STAT1 signaling is critically involved in the IFN-γ mediated enhancement of HIV-1 gp120 and Tat-induced neuronal damage. Primary cultured neuronal cells were co-treated with IFN-γ (100 U/ml) and gp120 or Tat at 250 ng/ml in the presence of JAK inhibitor (50 nM) for 12 h. Cell lysates were prepared for neuronal injury examination by Western blot analysis using Bc1-xL and Bax.

FIG. 9 is a graph depicting JAK/STAT1 signaling is critically involved in the IFN-γ mediated enhancement of HIV-1 gp120 and Tat-induced neuronal damage. Primary cultured neuronal cells were co-treated with IFN-γ (100 U/ml) and gp120 or Tat at 250 ng/ml in the presence of JAK inhibitor (50 nM) for 12 h. Cell lysates were prepared for neuronal injury examination by Western blot analysis using Bc1-xL and Bax. Data are presented as Western blot band density ratio of Bc1-xL to Bax (n=3). One-way ANOVA followed by post hoc comparison revealed significant differences between IFN-γ/gp120 or TFN-γ/Tat compared to JAK inhibitor/IFN-γ/gp120 or JAK inhibitor/IFN-γ/Tat (**P<0.001). Primary neuronal cells derived from STAT1-deficient mice were treated with gp120 or Tat at 250 ng/ml in the presence or absence of IFN-γ (100 U/ml) for 12 h. Cell cultured media and cell lysates from these cells were subjected to LDH assay.

FIG. 10 is a graph depicting JAK/STAT1 signaling is critically involved in the IPN-γ mediated enhancement of HIV-1 gp120 and Tat-induced neuronal damage. Primary neuronal cells derived from STAT1-deficient mice were treated with gp120 or Tat at 250 ng/ml in the presence or absence of IFN-γ (100 U/ml) for 12 h. Cell cultured media and cell lysates from these cells were subjected to LDH assay. Data are presented as the mean±SD of LDH release (n=5). One-way ANOVA followed by post hoc comparison revealed significant differences between STAT1-deficient neurons compared to wild-type neurons following treatment with IFN-γ/gp120 or IFN-γ/Tat for LDH release (P<0.001).

FIG. 11 is a blot depicting JAK/STAT1 signaling is critically involved in the IPN-γ mediated enhancement of HIV-1 gp120 and Tat-induced neuronal damage. Primary neuronal cells derived from STAT1-deficient mice were treated with gp120 or Tat at 250 ng/ml in the presence or absence of IFN-γ (100 U/ml) for 12 h. Cell cultured media and cell lysates from these cells were subjected to Western blot analysis.

FIG. 12 is a graph depicting JAK/STAT1 signaling is critically involved in the IPN-γ mediated enhancement of HIV-1 gp120 and Tat-induced neuronal damage. Primary neuronal cells derived from STAT1-deficient mice were treated with gp120 or Tat at 250 ng/ml in the presence or absence of IFN-γ (100 U/ml) for 12 h. Cell cultured media and cell lysates from these cells were subjected Western blot analysis. Data are presented as the Western blot band density ratios of Bc1-xL to Bax (n=5). One-way ANOVA followed by post hoc comparison revealed significant differences between STAT1-deficient neurons compared to wild-type neurons following treatment with IFN-γ/gp120 or IFN-γ/Tat for band density ratios of Bc1-xL to Bax (P<0.001).

FIG. 13 is a blot depicting EGCG inhibiting IFN-γ-induced JAK/STAT1 phosphorylation and protecting neurons from injury induced by IFN-γ/gp120 or IFN-γ/Tat in vitro. JAK1 protein phosphorylation was examined by Western blot. Cell lysates were prepared from primary cultured neurons treated with IFN-γ (100 U/ml) for various time points as indicated.

FIG. 14 is a blot depicting EGCG inhibiting IFN-γ-induced JAK/STAT1 phosphorylation and protecting neurons from injury induced by IFN-γ/gp120 or IFN-γ/Tat in vitro. Cell lysates were prepared from primary cultured neurons co-treated with IFN-γ (100 U/ml) and EGCG at different doses as indicated for 60 min.

FIG. 15 is a blot depicting EGCG inhibiting IFN-γ-induced JAK/STAT1 phosphorylation and protecting neurons from injury induced by IFN-γ/gp120 or IFN-γ/Tat in vitro. STAT1 protein phosphorylation was examined by Western blot. Cell lysates were prepared from primary cultured neurons treated with IFN-γ (100 U/ml) for various time points as indicated.

FIG. 16 is a blot depicting EGCG inhibiting IFN-γ-induced JAK/STAT1 phosphorylation and protecting neurons from injury induced by IFN-γ/gp120 or IFN-γ/Tat in vitro. Cell lysates were prepared from primary cultured neurons co-treated with IFN-γ (100 U/ml) and EGCG at different doses as indicated for 60 min.

FIG. 17 is a graph depicting EGCG inhibiting IFN-γ-induced JAK/STAT1 phosphorylation and protecting neurons from injury induced by IFN-γ/gp120 or IFN-γ/Tat in vitro. Cell cultured supernatants were collected for LDH assay. Data are presented as the mean±SD of LDH release (n=3). One-way ANOVA followed by post hoc comparison revealed significant differences between IFN-γ/gp120 or IFN-γ/Tat compared to EGCG/IFN-γ/gp120 or EGCG/IFN-γ/Tat for LDH release (**P<0.001).

FIG. 18 is a blot depicting EGCG inhibiting IFN-γ-induced JAK/STAT1 phosphorylation and protecting neurons from injury induced by IFN-γ/gp120 or IFN-γ/Tat in vitro. Cell lysates were prepared for Bc1-xL/Bax Western blot analysis.

FIG. 19 is a graph depicting EGCG inhibiting IFN-γ-induced JAK/STAT1 phosphorylation and protecting neurons from injury induced by IFN-γ/gp120 or IFN-γ/Tat in vitro. Cell lysates were prepared for Bc1-xL/Bax Western blot analysis. Data are presented as the Western blot band density ratios of Bc1-xL to Bax (n=3). One-way ANOVA followed by post hoc comparison revealed significant differences between IFN-γ/gp120 or IFN-γ/Tat compared to EGCG/IFN-γ/gp120 or EGCG/IFN-γ/Tat for band density ratio of Bc1-xL to Bax (**P<0.001).

FIGS. 20(A) through 20(D) are microscopy images demonstrating significant reductions in neuronal injury with EGCG treatment after i.c.v. injection of (A) IFN-γ/gp120, (B) IFN-γ/gp120/Tat, (C) IFN-γ/gp120/EGCG, or (D) IFN-γ/gp120/Tat/EGCG. Coronal, frozen mouse brain sections were stained with NeuN and analyzed for neuron injury/loss. A marked reduction of neuronal damage was observed when EGCG was added to either IFN-γ/gp120 or IFN-γ/gp120/Tat. Similar effects of EGCG were also observed in IFN-γ/Tat condition (data not shown).

FIG. 21 is a blot depicting Bc1-xL and Bax protein levels in mouse brain homogenates. The brain homogenates were treated with gp120/IFN-γ or gp120/IFN-γ/EGCG and analyzed by Western blot.

FIG. 22 is a blot depicting Bc1-xL and Bax protein levels in mouse brain homogenates. The brain homogenates were treated with Tat/IFN-γ or Tat/IFN-γ/EGCG and analyzed by Western blot.

FIG. 23 is a graph depicting Bc1-xL and Bax protein levels in mouse brain homogenates. Western blot data are presented as mean±SD of Western blot band density ratio of Bc1-xL to Bax (n=8; 4 female/4 male). One-way ANOVA followed by post hoc comparison revealed significant differences in the band density ratio of Bc1-xL to Bax observed between gp120/IFN-γ or gp120/Tat/IFN-γ compared to gp120/IFN-γ/EGCG or gp120/Tat/IFN-γ/EGCG conditions, respectively (**P<0.001).

### DETAILED DESCRIPTION OF THE PREFERRED EMBODIMENT

The term “adjuvant” as used throughout the specification to identify a pharmacological agent that modifies the effects of another pharmacological agent, irrespective of the direct effect of the adjuvant-agent.

The term “administration” or “administering” as used throughout the specification to describe the process in which a JAK1/STAT1 pathway inhibitor is delivered to a patient for treatment purposes. This includes parental, referring to intravenous and intraarterial as well as other appropriate parental routes; intrathecal; intraventricular; intraparenchymal; including spinal cord and brain stem; intracisternal; intracranial; intrastriatal; intranigral; and other routes that allow JAK1/STAT1 pathway inhibitor to contact neurons. The JAK1/STAT1 pathway inhibitor may be administered independently or in combination with other compounds, like antiviral compounds. Administration will often depend on the disease and level of progression in the afflicted brain.

The term antiviral treatment is used throughout the specification to identify a treatment combating the infectivity of viral infections, and specifically limiting retroviral virus load. The treatment includes highly active antiviral therapy (HAART), a combination of protease inhibitors developed in 1996. The treatment may include a combination of at least three drugs belonging to at least two anti-retroviral classes, including Nucleoside Analogue Reverse Transcriptase Inhibitors (NARTIs or NRTIs), protease inhibitors, and Non-nucleoside Reverse Transcriptase Inhibitors (NNRTIs). The term also encompasses daily multivitamin and mineral supplements which reduce viral progression.

The term “JAK1/STAT1” pathway is used throughout the specification to identify a cellular signaling pathway utilizing Janus Kinase (JAK) proteins and Signal Transducers and Activators of Transcrption (STAT) proteins. JAK proteins are tyrosine kinases which bind to cytokine receptors of a cell and, upon association of an extracellular ligand, become activated, phosphorylating phosphotyrosine-binding SH2 domains. STAT proteins, containing the SH2 domains, are activated and dimerize. Dimeric STAT proteins migrate into the nucleus activating transcription of target genes.

The term “neurodegenerative disease” is used throughout the specification to identify a disease which is caused by damage to the central nervous system and can be identified by neuronal death. Further, the term “neurodegenerative disease” as used herein describes “neurodegenerative diseases” which are associated with p-53 mediated cell cycle abrogation and apoptosis. Exemplary neurodegenerative diseases include HIV-associated Dementia, multiple sclerosis, Alzheimer's Disease, Parkinson's Disease, amyotrophic lateral sclerosis, and Pick's Disease.

The term “gp120” is used throughout the specification to identify a HIV viral surface protein and nucleic acid genetic code for the protein, existing in either RNA or DNA form. The protein is expressed on the surface of the viral envelope and binds to CD4 receptors of cells.

The term “Trans-Activator of Transcription”, “Tat” or “TAT” is used throughout the specification to identify a HIV viral transcription protein and nucleic acid genetic code for the protein, existing in either RNA or DNA form. The protein phosphorylates cellular factors in a host cell and may comprise between 86 and 101 amino acids.

The term “IFN-γ” is used throughout the specification to identify a type II interferon secreted by T-lymphocytes, NK cells, and dendritic cells. “IFN-γ” is a soluble cytokine that dimerizes, and has antiviral, immunoregulatory, and anti-tumor properties. “IFN-γ” acts via an interaction with a heterodimeric receptor consisting of IFNGR1 & IFNGR2 (interferon gamma receptors), thereby activating the JAK/STAT pathway.

The term “polyphenol” is used throughout the specification to identify is a group of chemical substances found in plants, characterized by the presence of more than one phenol group per molecule. “Polyphenols” include hydrolyzable tannins, lignins, flavonoids, and catechins.

### Experimental Procedures

Monoclonal mouse anti-neuronal nuclei antibody was obtained from Chemicon

(Temecula, Calif.). Donkey anti-mouse IgG Alexa Fluor 594 was purchased from Molecular Probes (Eugene, Or.). Tris-buffered saline was obtained from Bio-Rad (Hercules, Calif.) and luminol reagent was obtained from Pierce Biotechnology. Anti-phospho-STAT1/anti-phospho-JAK1, anti-total-STAT1/anti-total-JAK1, anti-Bc1-xL, and anti-Bax antibodies were purchased from Upstate (Lake Placid, N.Y.). Anti-actin antibody was obtained from Roche.

### In Vitro Neurotoxicity Analysis

Primary cultures of mouse cortical neurons were prepared as described previously (Chin et al., 1997). Briefly, neuronal cells were isolated from newborn C57BL16 mice and seeded in 6-well tissue-culture plates at 2×10⁵ cells/well for 48 h. Cells were then treated with gp120 (250 ng/ml) or Tat (250 ng/ml) in the presence or absence of IFN-γ (100 U/ml; Pierce Endogen) for 12 h. In addition, to test whether EGCG could inhibit JAK/STAT1 signaling and neuronal damage induced by gp120 or/and Tat in the presence of IFN-γ, EGCG was also employed as the co-treatment. Cell culture supernatants were used for LDH assay while cell lysates were used for Western blot analysis of Bc1-xL and Bax proteins.

### In Vivo Neurotoxicity Analysis

Animals were anesthetized using isoflurane (chamber induction at 4-5% isoflurane, intubation and maintenance at 1-2%). After reflexes were checked to ensure that mice were unconscious, they were positioned on a stereotaxic frame (Stoelting Lab Standard) with ear-bars positioned and jaws fixed to a biting plate. The axis coordinates were taken from a mouse brain atlas, and a 5-mm sterile plastic guide cannula (21 GA; Plastic One, Inc., Roanoke, Va.) was implanted into the left lateral ventricle delimited from the stereotaxic coordinates (coordinates relative to bregma: −0.6 mm anterior/posterior, +1.2 mm medial/lateral, and −3.0 mm dorsal/ventral) using the stereotaxic device (Stoelting Lab Standard) and an attached probe (cannula) holder. IFN-γ (200 U/mouse) with HIV-1 protein gp120 (500 ng/mouse) or Tat (500 ng/mouse) or PBS (10 μ) was administered at the rate of 1 μl/min using a Hamilton syringe (modified with a solder stop to prevent over insertion of the needle) through the implanted cannula. Correctness of the injection was confirmed by trypan blue dye administration and histological examination. The wounds were closed with 1-2 staples and mice were all observed until anesthesia had cleared. For testing EGCG effect on inhibiting Tat or/and gp120/IFN-γ neurotoxicity, the EGCG (50 mg/kg) or vehicle was intraperitoneally (i.p.) administered immediately after intracerebroventricular (i.c.v.) injection. Twenty-four hours after the i.c.v. injections animals were sacrificed with isofluorane and brain tissues collected. All dissected brain tissues were rapidly frozen for subsequent NeuN staining, Western blot, and LDH analysis.

### JAK/STAT1 Signaling Analyses

Normal C57BL/6 primary cultured neuronal cells as well as STAT1-deficient primary neuronal cells were isolated and cultured as described previously (Chin et al., 1997). Normal cells were co-treated with either gp120 or Tat (250 ng/ml) with or without IFN-γ (100 U/ml) and/or JAK inhibitor (50 nM). STAT1-deficient cells were treated with HIV-1 gp120 or HIV-1 Tat (250 ng/ml) in the presence or absence of IFN-γ (100 U/ml) for 12 h. At the end of the treatment period, neuronal cells were washed in ice-cold PBS three times and lysed in ice-cold lysis buffer. After incubation for 30 mm on ice, samples were centrifuged at high speed for 15 mm, and supernatants were collected. Total protein content was estimated by using the Bio-Rad protein assay. For phosphorylation of JAK1, membranes were first hybridized with phosphospecific Tyr1022/1023 JAK1 antibody (Cell Signaling Technology, Beverly, Mass.) and then stripped and finally analyzed by total JAK1. For STAT1 phosphorylation, membranes were probed with a phospho-Ser727 STAT1 antibody (Cell Signaling Technology), stripped with stripping solution, and then re-probed with an antibody that recognizes total STAT1 (Cell Signaling Technology). Alternatively, membranes with identical samples were probed either with phospho-JAK or STAT1 or with an antibody that recognizes total JAK or STAT1. Immunoblotting was performed with a primary antibody, followed by an anti-rabbit HRP-conjugated IgG secondary antibody as a tracer. After being washed in TBS, the membranes were incubated in the luminol reagent and exposed to film.

### LOFT Assay

LOU release assay (Promega, Madison, Wis.) was performed as previously described (Tan et al., 2002). Briefly, after treatment at a variety of conditions, cell cultured media were collected for LOU release assay. Total LDH release was represent maximal lysis of target cells with 5% Triton X-100. Data are presented as mean±SD of LOU release.

### Western Blot Analysis

Western blot was performed as described previously (Tan et al., 2002). Briefly, for the in vivo studies left hemispheres of mouse brains were lysed in ice-cold lysis buffer and an aliquot corresponding to 50 μg of total protein was electrophoretically separated using 16.5% Tris-tricine gels. Electrophoresed proteins were then transferred to PVDF membranes (BioRad), washed in dH₂O, and blocked for 1 h at ambient temperature in Tris-buffered saline containing 5% (w/v) nonfat dry milk. After blocking, membranes were hybridized for 1 h at ambient temperature with various primary antibodies. Membranes were then washed three times (5 mm each) in dH3O and incubated for 1 h at ambient temperature with the appropriate HRP-conjugated secondary antibody (1:1000). All antibodies were diluted in TBS containing 5% (w/v) non-fat dry milk. Blots were developed using the luminol reagent. Densitometric analysis was done using the Fluor-S Multilmager™ with Quantity One™ software (Bio-Rad). Antibodies used for Western blot included: anti-Bc1-xL antibody (1:1000), anti-Bax antibody (1:1000), anti-phospho-STAT1 (1:500), anti-total-STAT1 (1:500), anti-phospho-JAK1 (1:500), anti-total-JAK1 (1:500), and anti-actin antibody (1:1500). Similar procedures were followed for the in vitro studies using cell culture supernatant aliquots corresponding to 50 μg of total protein.

### NeuN Immunochemistry Analysis

At sacrifice, mice were anesthetized with isofluorane and transcardially perfused with ice-cold physiological saline containing heparin (10 U/ml). Brains were rapidly isolated and separated into left and right hemispheres using a mouse brain slicer (Muromachi Kikai, Tokyo, Japan). The right hemispheres were used for cryostate sectioning and subsequent NeuN immunochemistry analysis. NeuN staining was performed under standard immunofluorescence-labeling procedures. Briefly, frozen tissue sections were washed in PBS and blocked in 10% horse serum for 1 h, then incubated overnight in primary antibody, monoclonal mouse antineuronal nuclei antibody (1:100). The following day, sections were washed in PBS 3 times (10 mm each), and then incubated for 1 h in the dark with secondary antibody, donkey anti-mouse IgG Alexa Fluor 594 at 1:100. After another cycle of washing, floating sections were mounted onto slides, dehydrated and coverslipped with Vectashield fluorescence mounting media (Vector Labs., Burlingame, Calif.). Slides were visualized under dark field using an Olympus BX-51 microscopy.

### Statistical Analysis

All data were normally distributed; therefore, in instances of single mean comparisons, Levene's test for equality of variances followed by t-test for independent samples was used to assess significance. In instances of multiple mean comparisons, analysis of variance (ANOVA) was used, followed by post hoc comparison using Bonferonni's method. Alpha levels were set at 0.05 for all analyses. The statistical package for the social sciences release 10.0.5 (SPSS Inc., Chicago, Ill.) was used for all data analysis.

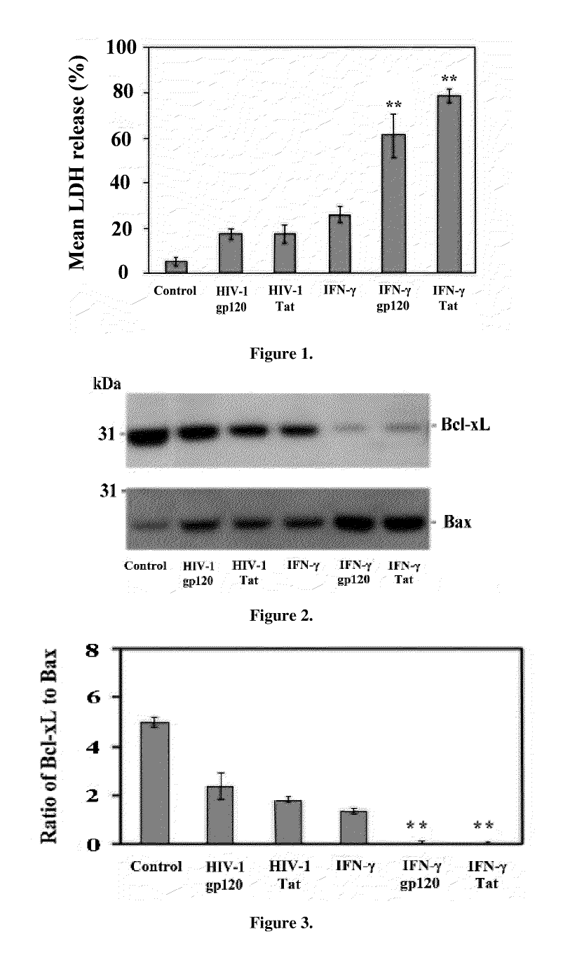

Collaboration of proinflammatory cytokines with HIV-1 proteins in the induction of neuronal injury/death appears to be an important component of the pathogenesis of HAD (Aquaro et al., 2005; Koenig et al., 1986; Xiong et al., 2000). Neurons express IFN-γ receptor (C. Bate et al., Interferon-gamma Increases Neuronal Death in Response to Amyloid-betal-42, J. Neuroinflammation, 28, 1-7, 2006), and IFN-γ receptor mRNA and protein are expressed by both neuron-like cells (N2a cells) and primary cultured neurons (data not shown). To test whether IFN-γ plays a role in the modulation of HIV-1 proteins gp120 and Tat-induced neuronal injury, primary cultured neuronal cells were isolated from newborn mice (1- to 2-day-old, wild-type C57BL/6; Jackson Laboratory, Bar Harbor, Me.) according to a method previously described (J. Tan et al., Role of CD40 Ligand in Amyloidosis in Transgenic Alzheimer's Mice, Nat. Neurosci., 5, 1288-1293, 2002). These cells were treated with gp120 or Tat (250 ng/ml) (recombinant HIV-1 proteins gp120 (HIV-1cN54 gp120) and Tat; The National Institutes of Health (NIH) AIDS Research and Reference Reagent Program, Rockville, Md.) in the presence or absence of IFN-γ (100 U/ml) (murine recombinant IFN-γ; R&D Systems, Minneapolis, Minn.) for 12 h. Cell cultured media were collected for LDH assay and cell lysates prepared for Western blot-based neuronal injury examination (Tan et al., 2002). The presence of IFN-γ significantly increased LDH release and reduced the band density ratio of Bc1-xL to Bax in primary neurons challenged with HIV-1 proteins gp120 or Tat, as seen in FIGS. 1 though 3, indicating IFN-γ enhances gp120 and Tat effects on cells as seen in FIGS. 1 and 2.

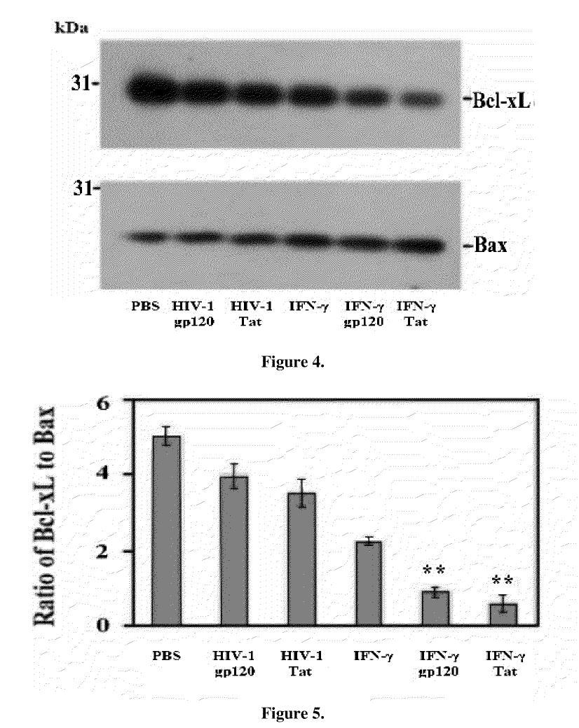

To test IFN-γ's ability to enhance neuronal injury induced by gp120 and Tat in vivo, C57B116 mice (n=8; 4 male/4 female) were treated with gp120 or Tat (500 ng/mouse) in the presence of IFN-γ (200 U/mouse) via intracerebroventricular (i.c.v) administration. Twenty-four hours after i.c.v. injection, the mice were sacrificed and brain tissues collected. All dissected brain tissues were rapidly frozen for subsequent biochemical and immunohistochemical analyses. Mouse brain sections from cortical regions were stained with NeuN and NeuN/DAPI. Results indicated a marked increase in neuronal damage in cortical brain regions from mice i.c.v injected with gp120 plus IFN-γ compared to controls, IFN alone, or gp120 alone, seen in FIGS. 4(A) through 4(D). (NeuN/DAPI results not shown). A similar result was also observed in the Tat plus IFN-γ condition (data not shown).

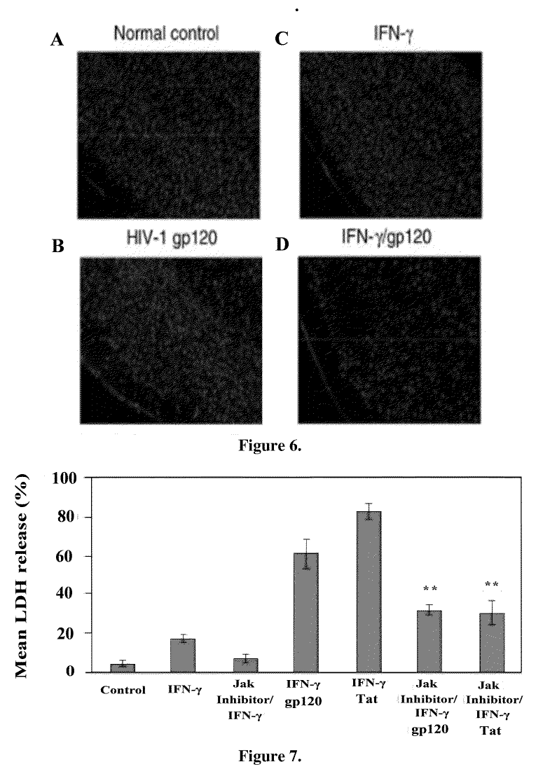

Brain homogenates from the mice were prepared for Western blot analysis of Bc1-xL and Bax protein expression. A significant reduction in the ratio of Bc1-xL to Bax was consistently observed with IFN-γ/gp120 or IFN-γ/Tat, seen in FIGS. 5 and 6. One-way ANOVA followed by post hoc comparison revealed significant differences between gp120 or Tat compared to gp120 or Tat plus IFN-γ for Western blot band density ratio of Bc1-xL to Bax. Thus, combining gp120 or Tat with IFN-γ results in a dramatic rise in neuron loss in the cortical regions, seen in FIGS. 5 and 6. Further, a synergistic, pro-apoptotic effect was observed when IFN-γ was combined with a challenge of HIV-1 gp120 or Tat proteins.

### EXAMPLE 1

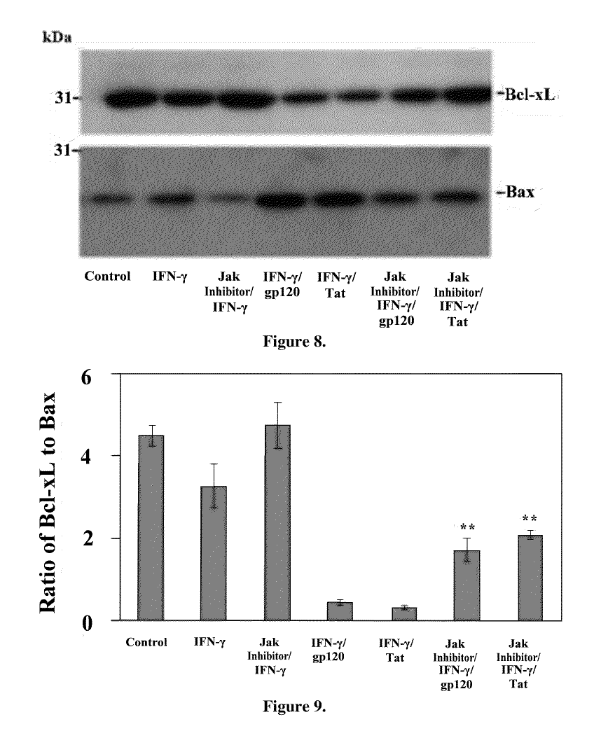

HIV infection of the CNS induces marked increases in IFN-γ expression in CNS tissues. Thus, IFN-γ-activated JAK/STAT1 signaling was analyzed to further investigate IFN-γ-enhanced neuronal injury induced by gp120 and Tat. Primary cultured neurons were treated with PBS, gp120 (250 ng/ml), Tat (250 ng/ml), IFN-γ (100 U/ml), and/or JAK inhibitor (50 nM) (2-(1,1-Dimethylethyl)-9-fluoro-3,6-dihydro-7H-benz[h]-imidaz [4,5-f] isoquinolin-7-one, EMD Biosciences, Inc., San Diego, Calif.) for 12 h. Neuronal injury was significantly inhibited by the presence of JAK inhibitor, as seen in FIGS. 7 through 9. One-way ANOVA followed by post hoc comparison revealed significant differences between IFN-γ/gp120 or IFN-γ/Tat compared to JAK inhibitor/IFN-γ/gp120 or JAK inhibitor/IFN-γ/Tat for both LDH release and Western blot band density ratio of Bc1-xL to Bax. I solated and cultured primary neurons from STAT1-deficient mice were treated with gp120 or Tat (250 ng/ml), in the presence or absence of IFN-γ (100 U/ml) for 12 h. Cell cultured media and cell lysates from these cells were then subjected to LDH and Western blot analyses. JAK1 inhibitor mitigated neuron damage, inflicted by combinations of IFN-γ and gp120 and Tat proteins, in vitro.

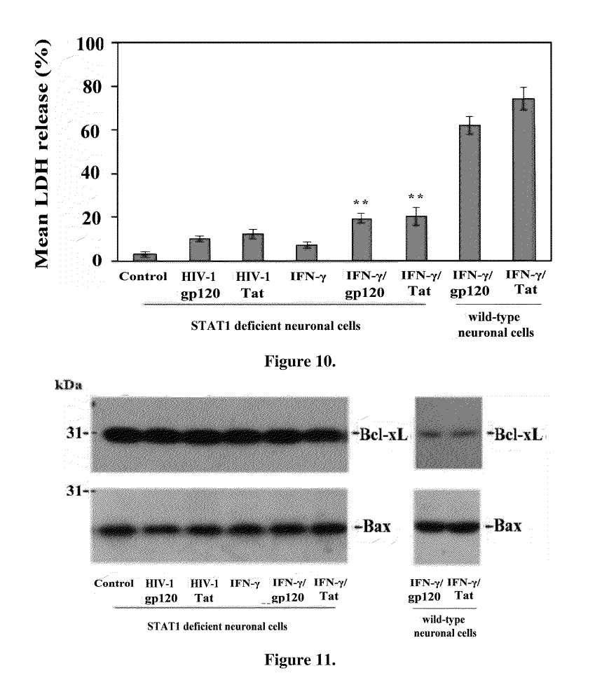

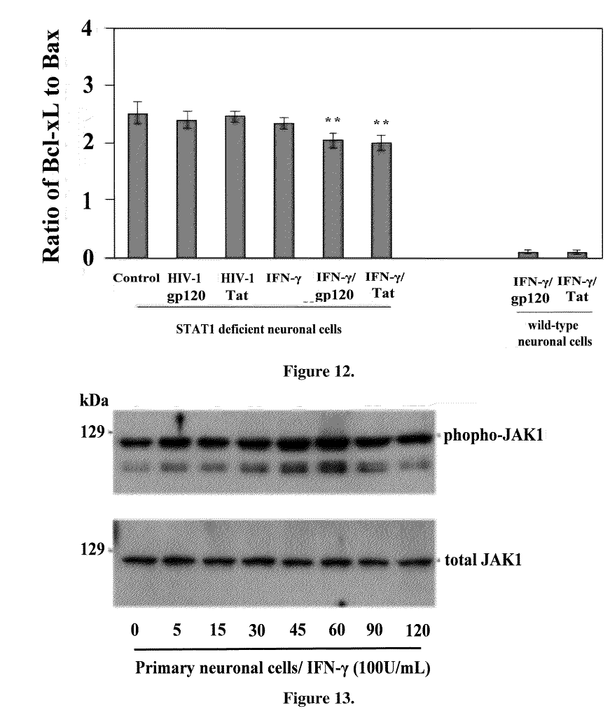

Both LDH and Bc1-xL/Bax ratios were greatly reduced by addition of JAK1 inhibitor. However, these cell death and apoptosis indicators did not return to baseline levels of the control treatment group when the combination of gp120 and Tat were included in the treatment; indicating an alternate pathway/mechanism utilized by these proteins to induce cell damage. Primary neurons from STAT1-deficient mice (Taconic, Hudson, N.Y.) were examined and found to be highly resistant to IFN-γ-enhanced neuron damage. Further, results demonstrated neuronal injury was largely attenuated in the STAT1-deficient neurons treated with IFN-γ/gp120 or IFN-γ/Tat. See FIGS. 10 through 12. One-way ANOVA followed by post hoc comparison revealed significant differences between STAT1-deficient neurons compared to wild-type neurons following treatment with IFN-γ/gp120 or IFN-γ/Tat for both LDH release and Western blot band density ratio of Bc1-xL to Bax.

### EXAMPLE 2

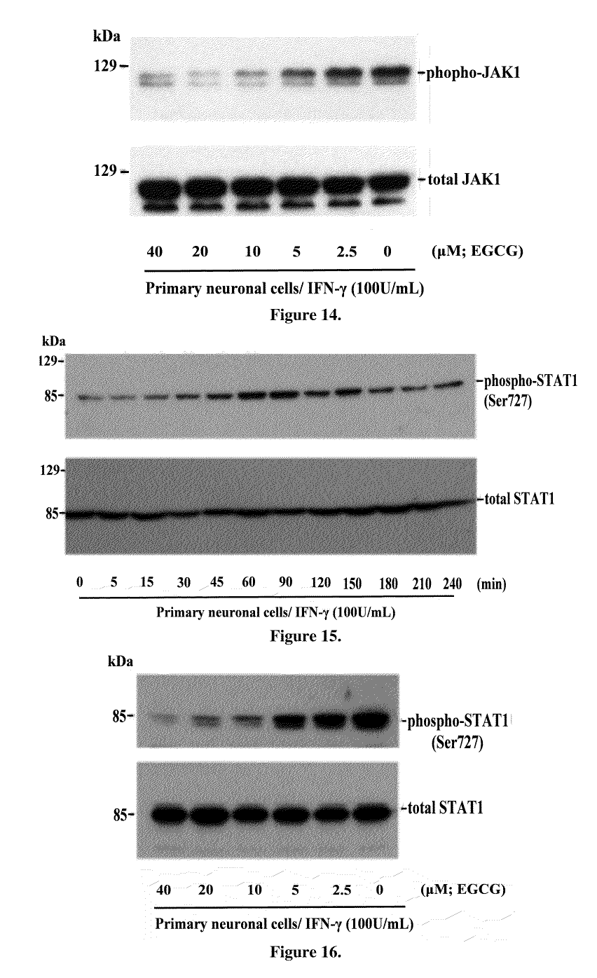

JAK1 and STAT1 activation was evident after treatment with IFN-γ in primary cultured neurons from wild-type mice, as seen in FIG. 13. The effect of JAK/STAT inhibition on neuronal damage was further analyzed using primary cultured neurons, treated with IFN-γ (100 U/ml) for different time points as indicated. Results demonstrated IFN-γ stimulates phosphorylation of JAK1, seen in FIG. 14, and STAT1, FIG. 15, time-dependently. To test whether EGCG could modulate this phosphorylation in neuronal cells, the cells were co-incubated with IFN-γ (100 U/ml) and EGCG (>95% purity by HPLC; Sigma, St. Louis, Mo.) at a range of doses as indicated for 60 min. JAK1/STAT1 phosphorylation was analyzed by Western blot. As shown in FIGS. 14 and 16, the presence of EGCG resulted in dose-dependent inhibition of JAK1/STAT1 phosphorylation.

### EXAMPLE 3

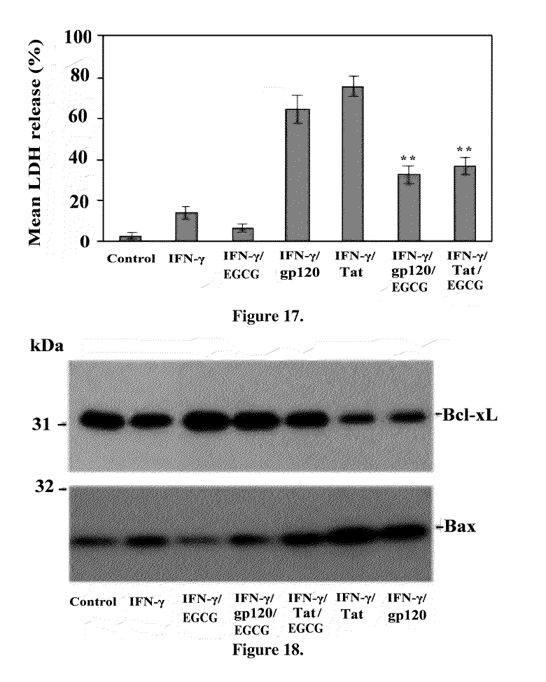

Green tea-derived polyphenol, EGCG, attenuates cell death induced by ischemia/reperfusion through downregulation of the JAK/STAT1 pathway (Townsend et al., 2004) and modulates STAT1 activation in vitro (de Prati et al., 2005; Magro et al., 2005; Tedeschi et al., 2002) and in vivo (Stephanou, 2004; Townsend et al., 2004). To examine the role of EGCG in opposing neuronal injury induced by HIV-1 gp120 or Tat in the presence of IFN-γ, primary neurons were co-treated with gp120 or Tat (500 ng/ml) in the presence of IFN-γ (100 U/ml) and EGCG (20 μM; >95% purity by HPLC; Sigma, St. Louis, Mo.) for 12 h. Cell culture supernatants were collected for LDH assay and cell lysates were prepared for Bc1-xL/Bax Western blot analysis. EGCG co-treatment markedly attenuates neuronal injury; as evidenced by decreased LDH release, seen in FIG. 17, and increased ratio of Bc1-xL to Bax, as seen in FIGS. 18 and 19. One-way ANOVA followed by post hoc comparison revealed significant differences between IFN-γ/gp120 or IFN-γ/Tat compared to EGCG/IFN-γ/gp120 or EGCG/IFN-γ/Tat for both LDH release and Western blot band density ratio of Bc1-xL to Bax. These data suggest that EGCG's ability to reduce JAK/STAT1 signaling in primary culture neurons is protective against IFN-γ-enhanced gp120/Tat-induced HAD-like neuronal damage in vitro.

### EXAMPLE 4

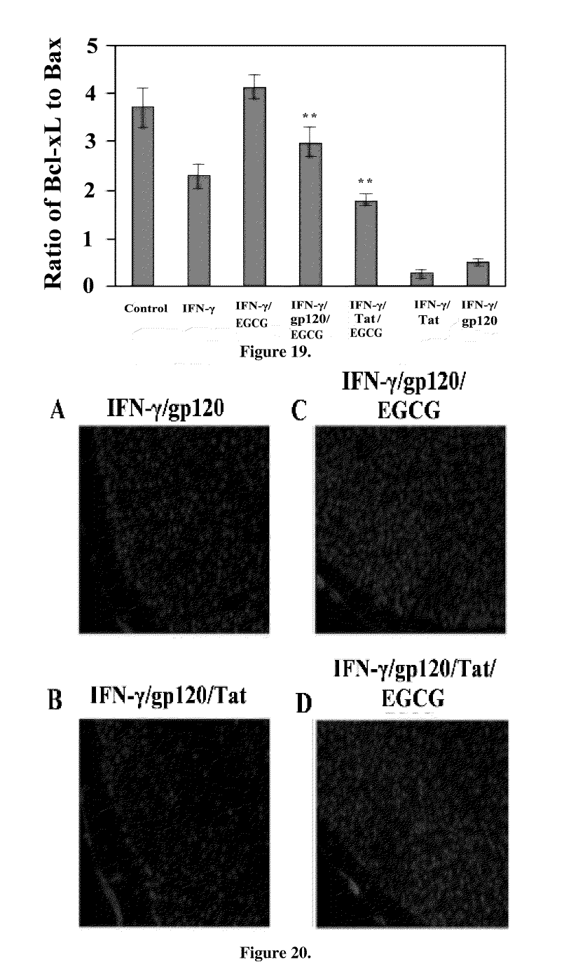

To evaluate EGCG's ability to inhibit neuronal damage induced by HIV-1 proteins in combination with IFN-γ in vivo, C57BL16 mice (n=8; 4 male/4 female) were treated with HIV-1 proteins gp120 or Tat (500 ng/mouse) in the presence of IFN-γ (200 U/mouse) via an i.c.v. injection. EGCG (50 mg/kg; >95% purity by HPLC; Sigma, St. Louis, Mo.) or vehicle was intraperitoneally (i.p.) administered immediately after the i.c.v. injection. Twenty-four hours after EGCG treatment, mice were sacrificed and brain tissues were rapidly frozen for biochemical and immunohistochemical analyses. Mouse brain sections from cortical regions were stained with NeuN and NeuN/DAPI. A marked reduction of neuronal damage was observed in cortical regions of the brains from mice i.c.v injected with IFN-γ/gp120 or IFN-γ/gp120/Tat in the presence of EGCG compared to controls, seen in FIG. 20(A) through 20(D). (NeuN/DAPI data not shown). Similar reductions in neuronal injury were also observed in mice treated with IFN-γ/Tat in the presence of EGCG compared to mice treated with IFN-γ/Tat alone (data not shown).

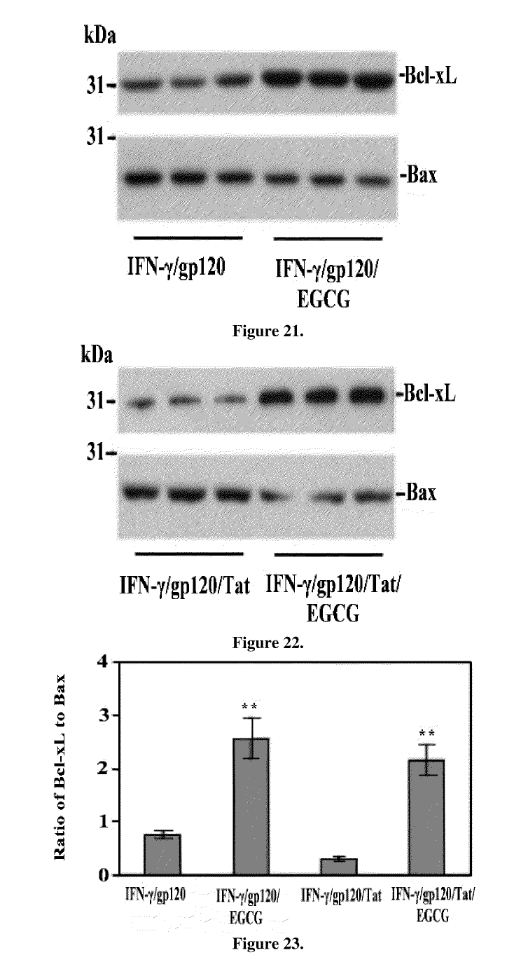

Brain homogenates were prepared from the mice for Western blot analysis of Bc1-xL and Bax protein expressions. Consistently, significant increases in the ratio of Bc1-xL to Bax were observed for both IFN-γ/gp120/EGCG and IFN-γ/gp120/Tat/EGCG, seen in FIGS. 21 through 23, compared to IFN-γ/gp120 and IFN-γ/Tat, respectively. One-way ANOVA followed by post hoc comparison revealed significant differences between IFN-γ/gp120/EGCG or IFN-γ/gp120/Tat/EGCG compared to IFN-γ/gp120 and IFN-γ/gp120/Tat in Western blot band density ratio of Bc1-xL to Bax. See, FIGS. 21 through 23.

EGCG was protective against neuron loss induced by i.c.v injected IFN-γ and/or gp120/Tat in cortical regions examined. This was evidenced by increased Bc1-xL/Bax ratios in brain homogenates of mice co-treated with EGCG plus IFN-γ/gp120 or IFN-γ/Tat/gp120, respectively, and reductions in neuron loss in cortical sections by immunohistochemistry.

Reports investigating EGCG's ability to block JAK/STAT1 signaling have reported protective effects of the compound against proinflammatory activation of immune cells, epithelial barrier dysfunction, and neuronal apoptosis after ischemia/reperfusion injury. Thus, JAK/STAT1 interaction may be an important therapeutic target for a variety of CNS disorders. However, the current data suggest the JAK/STAT1 pathway is an important therapeutic target for opposing the neuronal death and injury seen in the HAD brain. Indeed inhibition of the JAK/STAT pathway by green tea-derived EGCG or analogous compounds provides an effective therapeutic intervention as an adjunct to HAART for the treatment of HAD.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

What is claimed is:

1. A method of treating neurodegenerative disease comprising the steps of:
identifying a neurodegenerative disease caused by neuronal death; and
contacting neurons with an effective amount of a JAK/STAT pathway inhibitor.

2. The method of claim 1, wherein the neurodegenerative disease is selected from
the group consisting of HIV-associated Dementia, multiple sclerosis, Alzheimer's Disease, Parkinson's Disease, amyotrophic lateral sclerosis, and Pick's Disease.

3. The method of claim 2, wherein the neurodegenerative disease is HIV-associated Dementia.

4. The method of claim 3, wherein the HIV-associated Dementia is caused by gp120 or Tat protein.

5. The method of claim 4, wherein the gp120 or Tat protein is extracellular within the brain.

6. The method of claim 2, wherein the neurodegenerative disease is IFN-γ-enhanced.

7. The method of claim 1, wherein the neurodegenerative disease is caused by activation of the JAK1/STAT1 pathway.

8. The method of claim 1, wherein the JAK/STAT pathway inhibitor modulates JAK1 phosphorylation.

9. The method of claim 1, wherein the JAK/STAT pathway inhibitor is a tea-derived polyphenol.

10. The method of claim 9, wherein the polyphenol is a catechin.

11. The method of claim 10, wherein the tea-derived catechin is EGCG.

12. The method of claim 11, wherein EGCG is administered to a patient with a neurodegenerative disease at a concentration of between 5 μM-40 μM.

13. The method of claim 11, wherein EGCG is administered to a patient with a neurodegenerative disease at a concentration of between 10 μM-40 μM.

14. The method of claim 11, wherein EGCG is administered to a patient with a neurodegenerative disease at a concentration of 20 μM.

15. The method of claim 11, wherein EGCG is administered after HIV proteins have been identified in the brain.

16. The method of claim 1, wherein the JAK/STAT pathway inhibitor is an adjuvant to an antiviral treatment.

17. The method of claim 14, wherein the antiviral treatment is HAART.

18. A method for simulating neuron death-related dementia comprising the steps of:
contacting neuronal cells with a compound selected from the group consisting of HIV-1 gp120, HIV-1 Tat, gp120, Tat, and gp120 and Tat; and
contacting neuronal cells with IFN-γ.

19. The method of claim 16, wherein the neuron death-related dementia is HIV-associated Dementia.
